(19) United States
(12) Patent Application Publication
(10) Pub. No.: US 2024/0132361 A1
(43) Pub. Date: Apr. 25, 2024

(54) THERMAL TREATMENT OF COKE PRODUCED FROM CARBON OXIDES

(71) Applicant: Seerstone LLC, Provo, UT (US)

(72) Inventor: Randall Smith, Sandy, UT (US)

(73) Assignee: Seerstone LLC, Provo, UT (US)

(21) Appl. No.: 17/769,234

(22) PCT Filed: Oct. 16, 2020
(86) PCT No.: PCT/US20/56070

§ 371 Date: Apr. 14, 2022

Related U.S. Application Data
(60) Provisional application No. 62/926,978, filed on Oct. 28, 2019.

(51) Int. Cl. C01B 32/205; C07C 1/12

(52) U.S. Cl. CPC C01B 32/205; C07C 1/12; C01P 2004/04; C01P 2006/12

(57) ABSTRACT

A carbon pitch or dry coke is produced by reacting a mixture of carbon dioxide and hydrogen in a reactor at a predetermined temperature and pressure with an iron catalyst being fed into the reactor, using a process that also involves production of methane from the reaction gases. The reaction product is cooled. The reaction product may be graphitized in a reaction vessel under reduced pressure, heating the vessel at a predetermined rate and injecting an inert gas flow. The vessel is heated to a temperature of between 1600° C. and 2800° C. and maintained at that temperature for hours. The vessel is cooled and the reaction products removed.

### CROSS-REFERENCE TO RELATED APPLICATION

This application claims the benefit of the filing date of U.S. Provisional Patent Application Ser. No. 62/926,978 filed 28 Oct. 2019 and titled “Process for Making Synthetic Graphite from Carbon Oxides,” the disclosure of which is incorporated herein in its entirety by this reference.

### BACKGROUND

Various methods for producing carbon of different morphologies exist. For example, U.S. Pat. No. 8,679,444, the specification of which is incorporated herein by this reference, discloses a novel method of producing carbon fiber (the “Noyes Process”), carbon nanotubes, amorphous carbon, and other morphologies. That disclosure has been expanded because of the production of nano-diamonds (see U.S. Pat. No. 9,475,699, the specification of which is incorporated herein by this reference) and other morphologies of carbon.

Because of the benefits of carbon capture and carbon production cost when using the Noyes Process, it would be useful to be able to adapt the Noyes Process in such a way as to produce coke materials from carbon oxides (for example, carbon monoxide and carbon dioxide). Previously, such coke production methods passed through a liquid phase, see H. Marsh Introductions to Carbon Science, Chapter 1, page 1, 3^{rd} paragraph: “The former (cokes) come from carbonaceous precursors which pass through a liquid phase on pyrolysis (e.g. pitches).” The Noyes Process C—H—O equilibrium diagram, see U.S. Pat. No. 8,679,444, shows the relationship between the hydrocarbon pyrolysis, Boudouard and Bosch Reactions.

The Boudouard and Bosch processes, under the proper conditions (i.e. temperature, catalyst, pressure, and gas composition), typically produce anisotropic carbons. This carbon is a highly disordered carbon, see FIG. 16, and may be graphitizable, see FIG. 36; see also, H. Marsh definition of graphitizable carbon, page 30, conclusion “Graphitizable carbons have passed through a fluid phase during pyrolysis.” At no point does the Boudouard or Bosch process pass through a fluid phase.

Carbon produced using the Noyes Process typically contains iron or other catalytic material, because of use of iron or other metals to catalyze the reaction. For some uses, it is better that this iron be removed from the carbon product. Also, there are benefits to producing carbon with lower surface area or having tighter construction. Thus, it would be helpful if there were a method for taking carbon and creating products with different properties.

### SUMMARY

According to the present disclosure, a thermal treatment process imparts different properties to carbon feedstocks. The carbon feedstock might be Noyes Process carbon (typically this is carbon nanotubes and carbon fibers with some amorphous or perhaps graphitic carbon as well), or other carbon morphologies. At present, it appears that various morphologies of carbon could be used, including naturally occurring graphitic carbon, other synthetically produced carbon (including carbon fiber, nanotubes, graphite, and amorphous carbon), and other carbon sources.

One method of producing the carbon pitch to be used for graphitization involves making a coking material. According to this process, a hydrogen and CO₂ mixture of reaction gases are heated and injected into a reactor. Typically, the presence of the nickel in the metallurgy of the piping used in the construction of the heat exchanger instigates a Sabatier process reaction, in which some of the CO₂ and the H₂ react to form methane. Inside the reactor, a catalyst material of iron, nickel, chromium, or other metals or alloys of metals causes the CO₂ and the H₂ to react at temperatures of approximately 340° C. and 715° C., at which temperatures the carbon oxides and methane are converted to solid carbons and water in the presence of the catalyst. The result is often a blend of graphitic carbons and pyrolytic carbons. The ratio of these carbons can be varied by controlling the methane percentage within the reactor. Pyrolytic carbons are formed by the conversion of methane to solid carbon and hydrogen in this portion of the reaction. Graphitic carbons are produced by the Bosch reaction. A catalyst feeder deposits the catalyst into the reactor.

As the carbons are formed within the reactor vessel, various morphologies can be produced by controlling the residence time in the reactor, for example, by converting carbon fiber to coke and blends thereof. Residence time is controlled by the flow rate of reaction gases through the reactor. The resulting carbon products are then carried out of the reactor. The reaction gases are cooled and water is condensed out of the reaction gases. The resulting carbon (carbon pitch or coke) may then be used for the graphitization process.

According to one embodiment of the method of the present disclosure, the coke or carbon pitch is placed in a crucible or other container made of a material that can withstand the temperatures involved in the method. The carbon and container are placed in a vacuum furnace, the vacuum pump turned on, and the furnace is gradually brought to the desired temperature. For example, the furnace temperature may be raised by 20° C. every minute until the treatment temperature (which is typically in excess of 1500° C.) is reached. In some embodiments, the vacuum pump is then turned off and a helium flow (or other relatively inert gas, such as nitrogen, argon, or neon) is passed through the furnace. In other embodiments, no gaseous flow is used.

The furnace is maintained at the desired elevated temperature, often for several hours. The furnace is then cooled, and the container removed. In the experiments described below, the resulting carbon was found to have significantly different properties than the original carbon before treatment. For example, the resulting carbon was tested and found to have significantly greater thermal and electrical conductivity, more consistent D spacing, and lower surface area, as well as containing less iron, in some experiments, significantly less iron.

Furthermore, examination of TEM images of the product showed that the resulting product had significantly “tighter” spacing. That is, the carbon atoms appear to be much better aligned in a graphitic pattern.

The present process differs from the earlier Noyes Process in part because of the use of a different catalysts and different gas feed rates for the reaction. For example, the present process may employ FeC, Fe₂O₃, and Fe₃O₄, rather than elemental iron. The gas feed rates also differ as will be addressed in the experimental processes explained below. However, the end result is that the carbon from carbon oxides may be captured or sequestered into the form of coke or carbon pitch. Production of large amounts of coke at competitive rates will greatly assist in steel production. In fact, the present process would permit a steel plant to take carbon oxide emissions from the steel plant, convert the carbon oxides into elemental carbon, and then put the carbon back into blast furnace (in the form of the coke), and thus incorporate the captured carbon oxides into the steelmaking process (again, in the form of coke).

### BRIEF DESCRIPTION OF THE DRAWINGS

FIGS. 1-8 depict SEM images of the carbon feedstock used in the presently disclosed experiments;

FIG. 9 depicts an energy dispersive spectroscopy (“EDS”) graph and chart showing the iron percentage of the samples depicted in FIGS. 1-8;

FIGS. 10-16 depict TEM images of the carbon feedstock prior to thermal treatment;

FIGS. 17 and 18 depict graphs of the inner and outer D spacing of the carbon feedstock shown in FIG. 13;

FIGS. 19 and 20 depict graphs of the inner and outer D spacing of the carbon feedstock shown in FIG. 16;

FIGS. 21-24 depict TEM images of the product from the 1600° C. thermal treatment;

FIGS. 25 and 26 depict graphs of the inner and outer D spacing of the carbon product shown in FIG. 23;

FIGS. 27-30 are TEM images of the product from the 2000° C. thermal treatment;

FIGS. 31 and 32 depict graphs the inner and outer D spacing of the 2000° C. treated feedstock depicted in FIG. 30;

FIGS. 33-36 depict TEM images of the product from the 2400° C. thermal treatment;

FIGS. 37 and 38 depict graphs the inner and outer D spacing of the 2400° C. treated feedstock depicted in FIG. 36;

FIG. 39 shows the surface area, density, conductivity, resistivity, EDS (energy dispersive spectroscopy), and TGA (thermographic analysis) for each of the three experimental products; and

FIG. 40 depicts a schematic representation of an exemplary process flow diagram.

### DETAILED DESCRIPTION

The present disclosure involves the effects of thermally treating carbon for removing iron and changes to the characteristics of the carbon properties by thermally annealing material from a 0.3-ton reactor at different temperatures. In this case, the 0.3 ton reactor is used to make carbon of various morphologies. However, the present process should work with other carbon morphologies, including those created using the iron acetate catalytic process disclosed in U.S. Provisional Patent Application Ser. No. 62/444,587, the disclosure of which is incorporated herein by this reference.

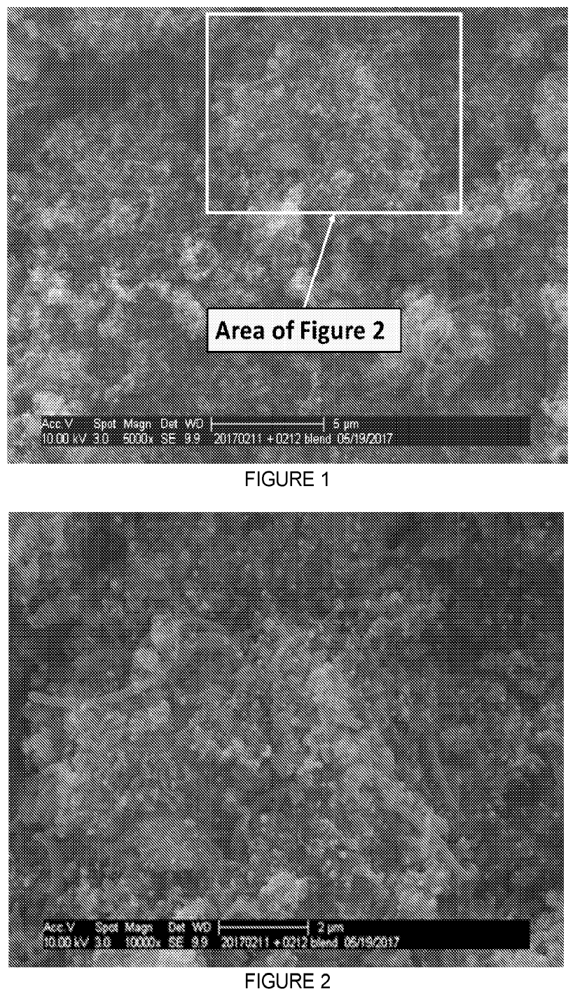

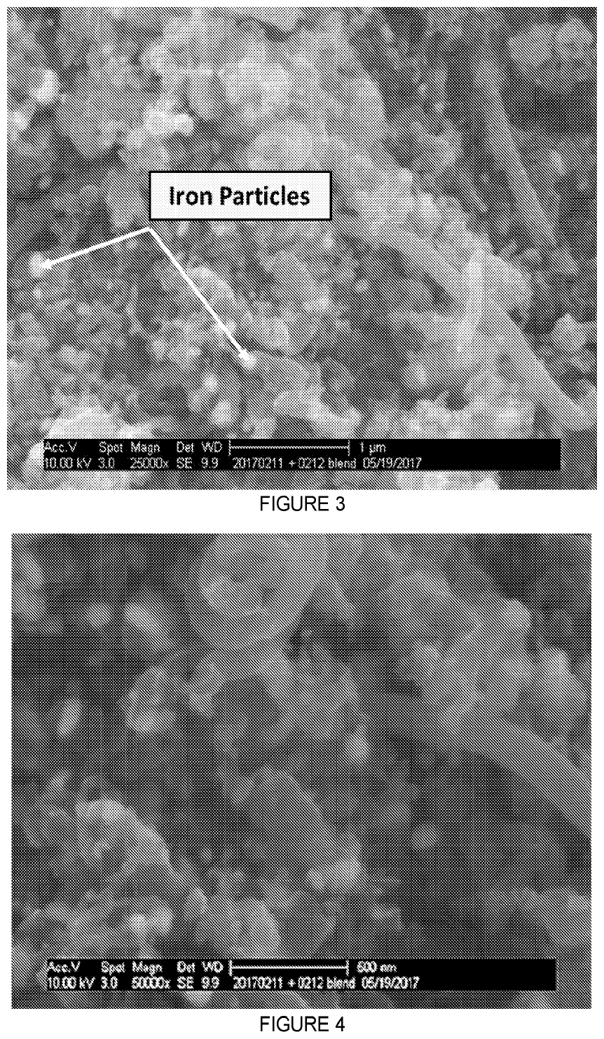

FIGS. 1-8 depict SEM images of the carbon feedstock used in the present experiments. As indicated, FIG. 1 is at a magnification of 5000×. FIG. 2 depicts a portion of the material depicted in FIG. 1, but at a 10,000× magnification. FIG. 3 depicts the feedstock carbon at 25,000× magnification. Small iron particles are highlighted in FIG. 3. FIG. 4 depicts the feedstock at 50,000× magnification.

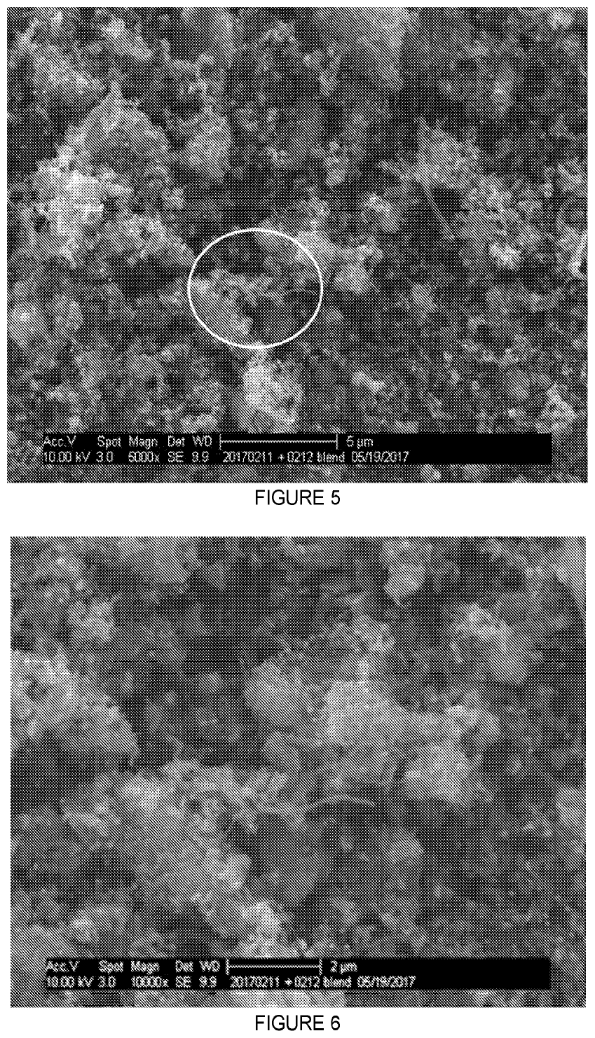

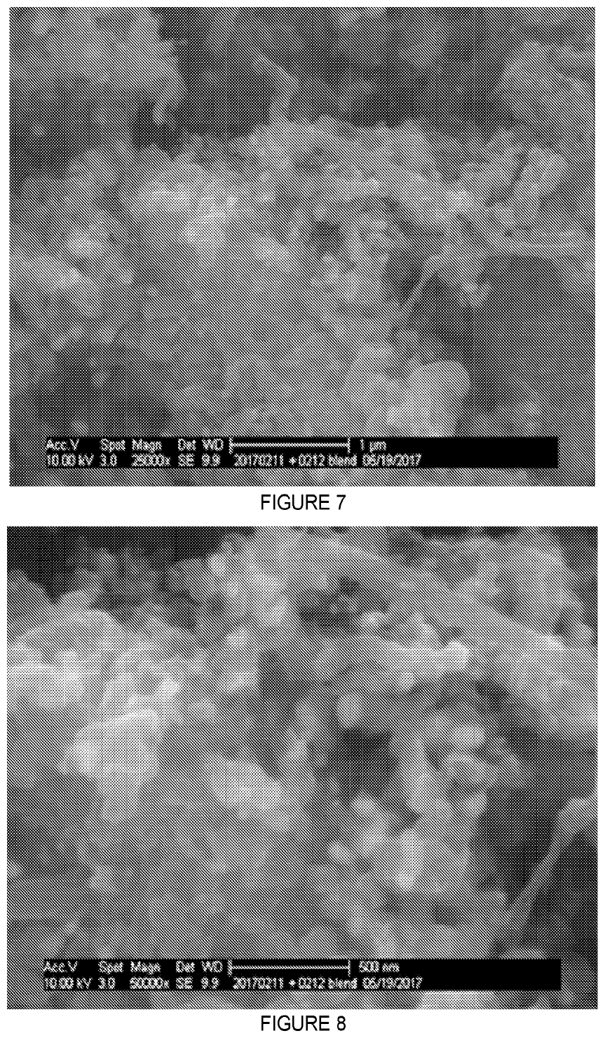

FIG. 5 depicts a different portion of the feedstock at 5000×. FIG. 6 is of the identified portion of FIG. 5, but at 10,000× magnification. FIGS. 7 and 8 are close up images of the feedstock depicted in FIGS. 5 and 6 at 25,000× and 50,000× magnification, respectively.

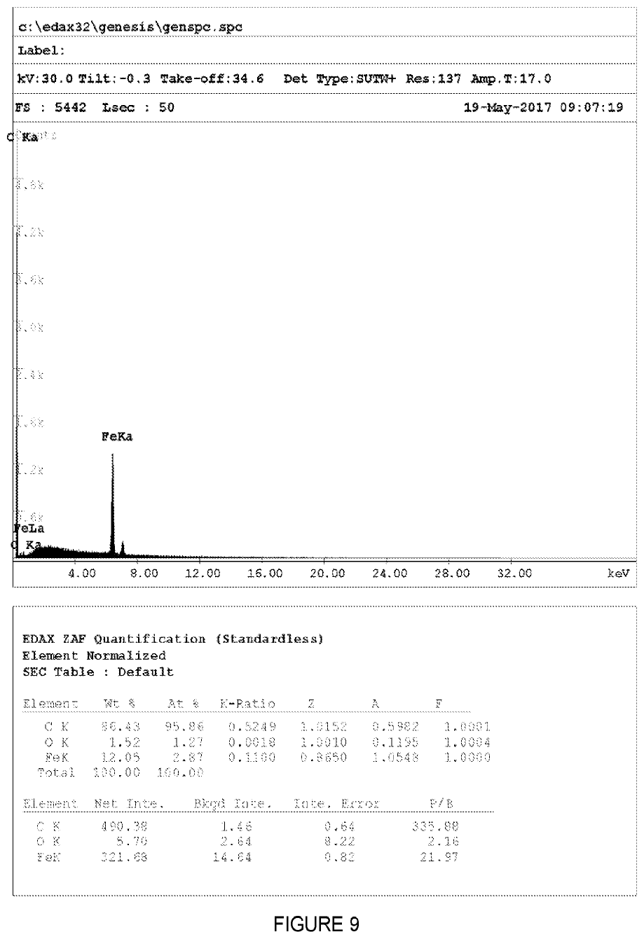

FIG. 9 depicts an energy dispersive spectroscopy (“EDS”) graph and chart showing the iron percentage of the samples depicted in FIGS. 1-8. As indicated therein, the iron content of the feedstock carbon was in excess of 12%. If the carbon is intended for use as coke, such an iron content is acceptable. However, if the sample is to be thermally treated to form graphite, a part of the thermal treatment assists in reducing this iron content.

The K-ratio that is referenced in FIG. 9 is calculated by measuring the x-ray intensity ratio k=I_{unknown}/I_{standard} for each element and calculating concentrations by applying matrix corrections for electron backscattering and energy loss (Z), x-ray absorption (A), and characteristic- and continuum-induced secondary x-ray fluorescence (F), as originally developed for the electron probe microanalyzer (EPMA) with wavelength dispersive spectrometry (WDS). By utilizing the K-ratio protocol, SEM/EDS has been shown to be capable of matching the precision and accuracy of EPMA/WDS for major (concentration C>0.1 mass fraction) and upper trace range (0.001<C<0.01) constituents, even when significant peak interference occurs.

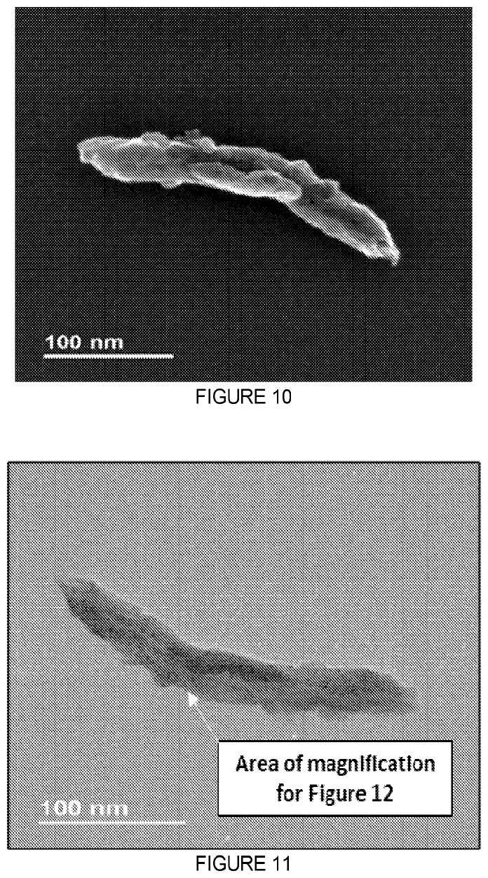

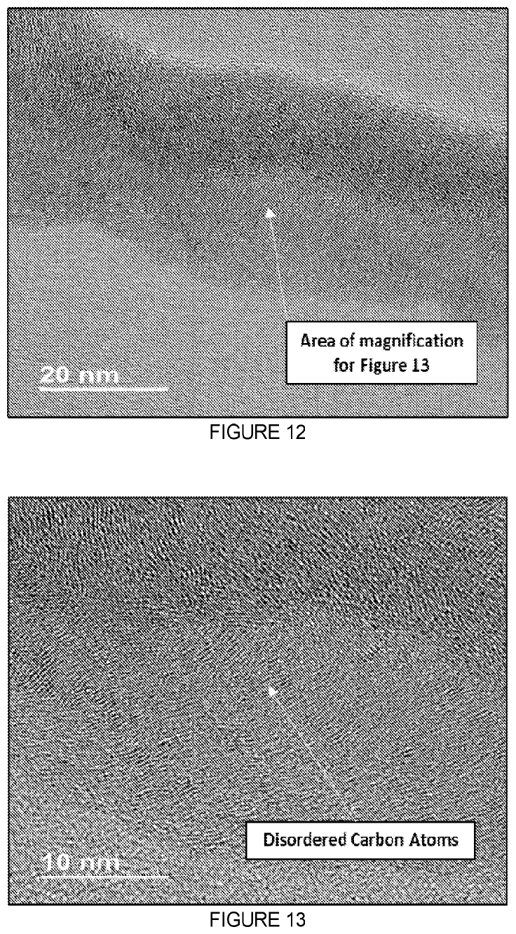

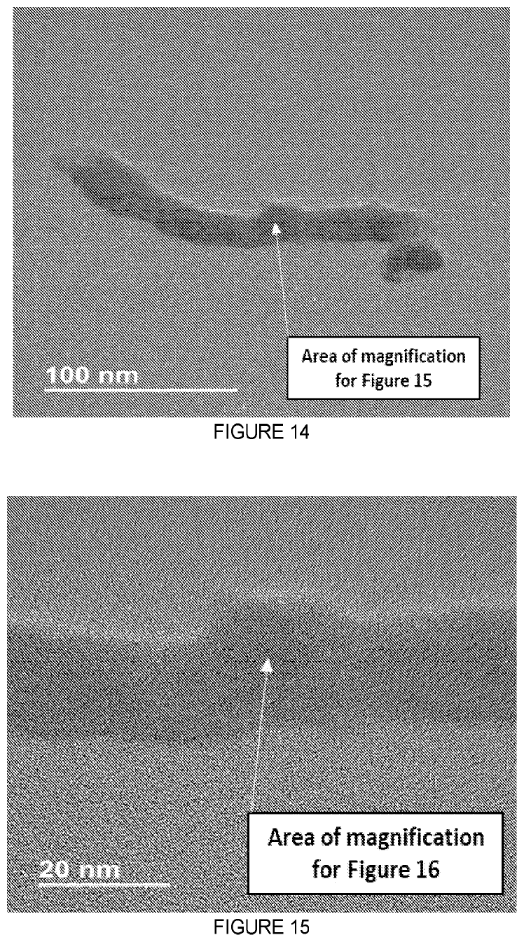

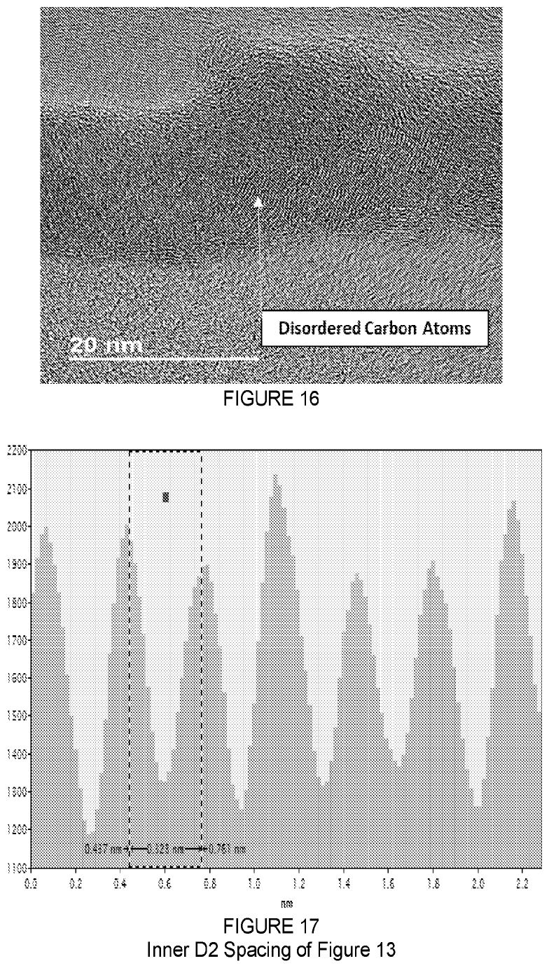

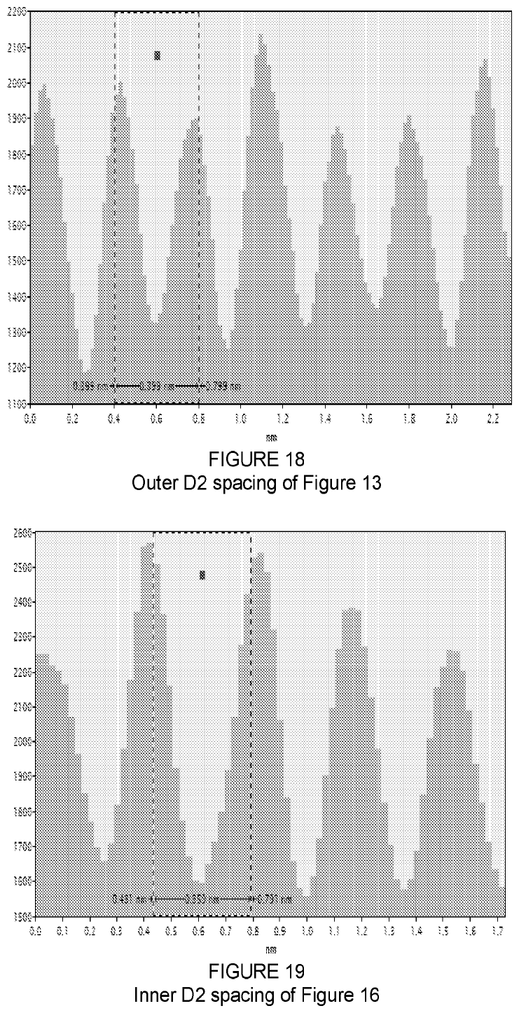

FIGS. 10-13 depict the carbon feedstock prior to thermal treatment. Close examination of these images, and in particular FIGS. 12 and 13, show the small piece of the carbon feedstock depicted has relatively disordered carbon atoms. That is, the “lines” of carbon atoms are disjointed, disconnected, and not particularly linear, except for short stretches. Close examination of FIGS. 14-16, which also depict pre-treated carbon feedstock, shows the same properties.

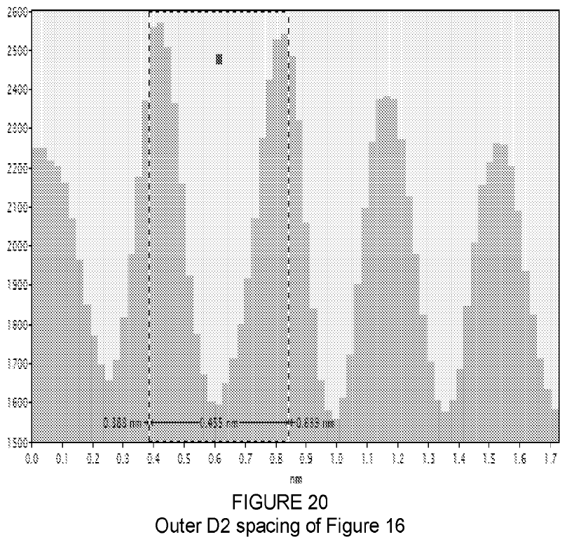

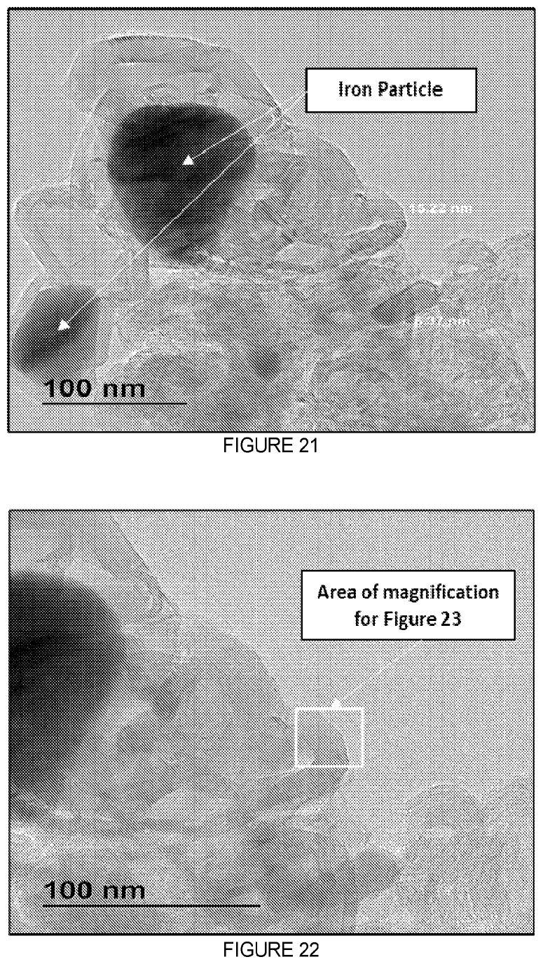

The D spacing of the feedstock carbon depicted in FIG. 13 is graphed in FIGS. 17 and 18. FIGS. 19 and 20 graph the D spacing for the carbon feedstock depicted in FIG. 16.

Thermal Treating Process

Different samples of carbon (from the 0.3 ton per month reactor, mentioned above) were subjected to a process of thermal treatment. Three such experiments are briefly described below. The three experiments involved high-end temperatures of 1600° C., 2000° C., and 2400° C.

In each case, the process involved placing 30 grams of the carbon feedstock into a graphite crucible (graphite being used as it is known to be able to withstand the temperatures involved). The crucible and feedstock were placed into a vacuum furnace, which was then closed, and the vacuum pump started. The furnace temperature was increased by approximately 20° C. per minute.

In the first two experiments (1600° C. and 2000° C.), the feedstock was left in the furnace for 4 hours at the designated temperature. The furnace was then cooled to 150° C., and the furnace opened, allowing the material to cool to room temperature. The material was then removed from the furnace.

As outlined below, for the 2400° C. experiment, upon reaching a temperature of 2000° C., the furnace was held at that temperature long enough to thermally stabilize at that temperature. The vacuum pump was turned off and a helium flow of 5 standard cubic feet per minute through the furnace began. The furnace temperature was again increased, at approximately 20° C. per minute, until the internal temperature reached 2400° C. The furnace was held at 2400° C. for 4 hours, and the furnace was then cooled to 150° C., opened to allow the furnace to return to room temperature, and the material was removed.

In each of the experiments, after reaching a set temperature, the vacuum pump was turned off and a slow gas flow (approximately 5 cubic feet per second) was injected into the furnace. The gases used were typically nitrogen up to a temperature of about 2000° C., argon from 2000° C. to about 2400° C., and helium above 2400° C. The experiments were conducted as described below, with the high-end temperature used as a caption, and the steps of the experiments given in a numbered list:

1600° C.

| | |
|---|---|
| 1. | Load 30 gm of material into graphite crucible. |
| 2. | Place crucible into vacuum furnace. |
| 3. | Close furnace. |
| 4. | Start vacuum pump. |
| 5. | Start furnace heater element. |
| 6. | Start argon flow. |
| 7. | Ramp furnace at a rate of 20° C. minute. |
| 8. | Hold at 1600° C. for 4 hours. |
| 9. | Let furnace cool to 150° C. |
| 10. | Open furnace and allow crucible cool to room temperature. |
| 11. | Remove material from crucible. |

2000° C.

| | |
|---|---|
| 1. | Load 30 gm of material into graphite crucible. |
| 2. | Place crucible into vacuum furnace. |
| 3. | Close furnace. |
| 4. | Start vacuum pump. |
| 5. | Start furnace heater element. |
| 6. | Start argon flow, |
| 7. | Ramp furnace at a rate of 20° C. minute. |
| 8. | Hold at 2000° C. for 4 hours. |
| 9. | Let furnace cool to 150° C. |
| 10. | Open furnace and allow crucible cool to room temperature. |
| 11. | Remove material from crucible. |

2400° C.

| | |
|---|---|
| 1. | Load 30 gm of material into graphite crucible. |
| 2. | Place crucible into vacuum furnace. |
| 3. | Close furnace. |
| 4. | Start vacuum pump. |
| 5. | Start furnace heater element. |
| 6. | Ramp furnace at a rate of 20° C. minute. |
| 7. | Hold at 2000° C., hold [long enough to stabilize at temp - 30 mins |
| | nominal], and start helium flow at 5 scfh [std ft3/hr]. |
| 8. | Turn off vacuum pump. |
| 9. | Ramp furnace to 2400° C. at 20° C. a minute. |
| 10. | Hold at 2400° C. for 4 hours. |
| 11. | Let furnace cool to 150° C. |
| 12. | Open furnace and allow crucible cool to room temperature. |
| 13. | Remove material from crucible. |

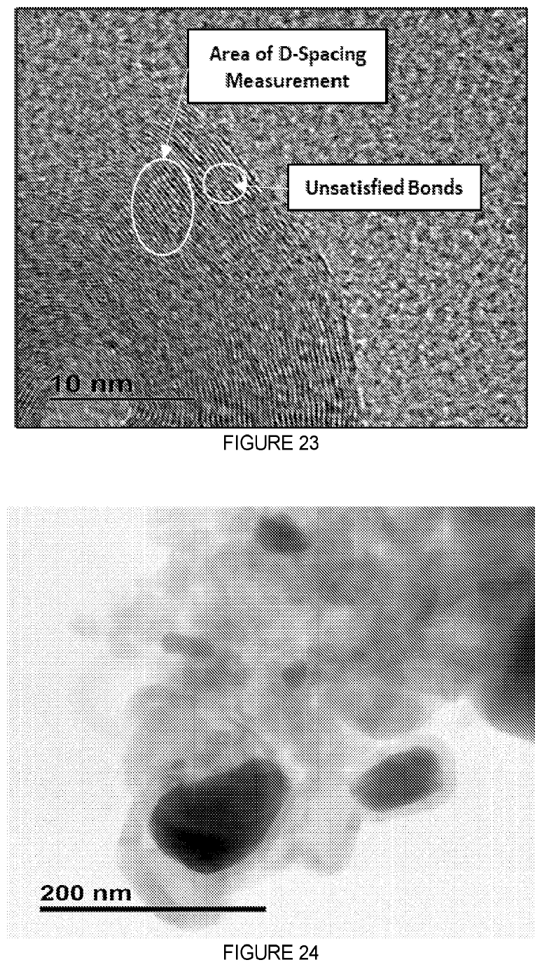

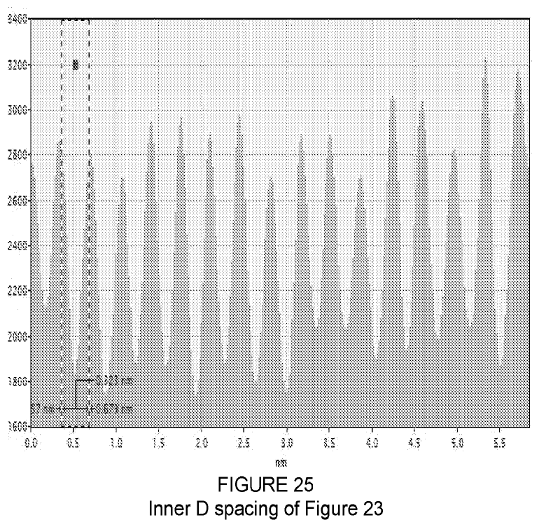

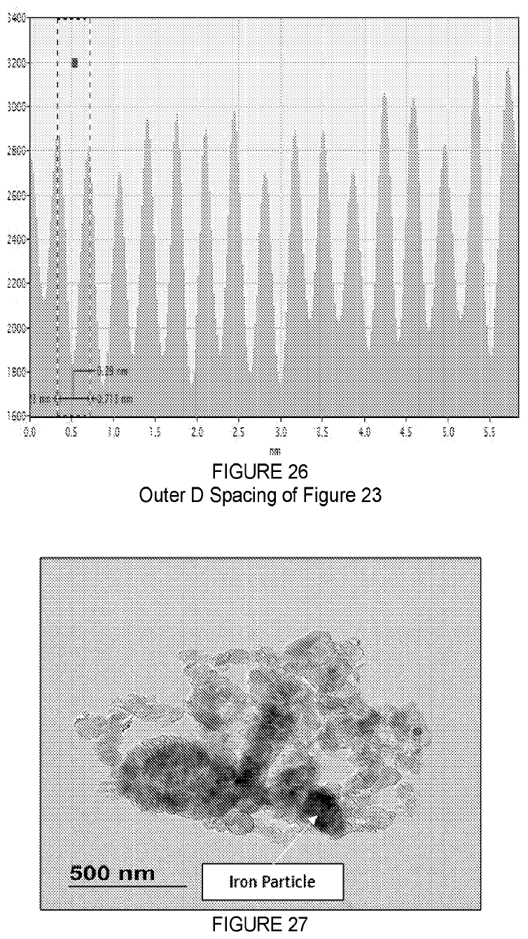

FIG. 23, which is taken from the identified portion of the product depicted in FIG. 22, shows how ordered the carbon atoms have become. Note the lines of graphitic carbon, showing significantly more ordered atomic carbon than the original carbon feedstock had.

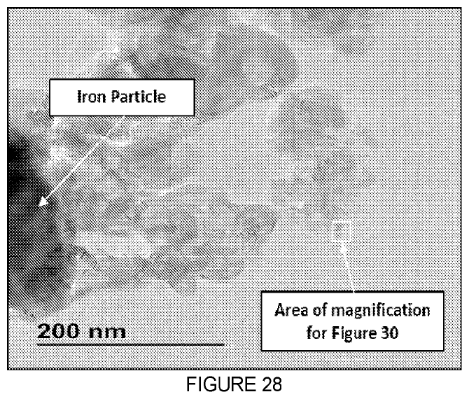

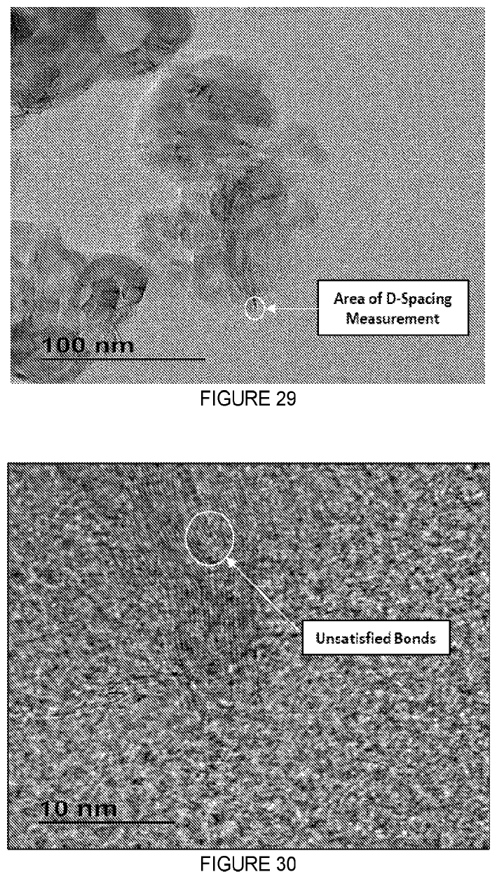

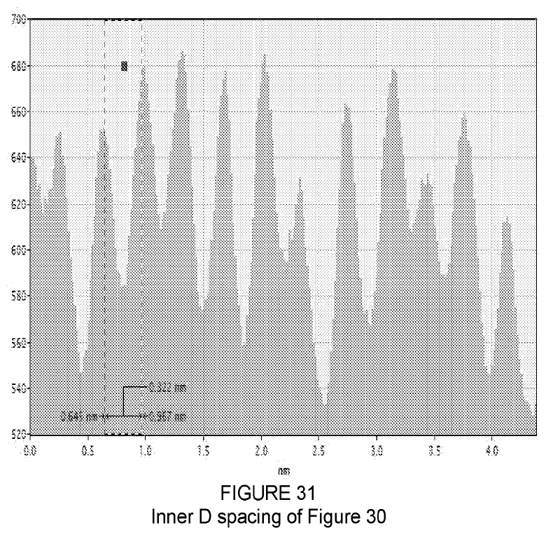

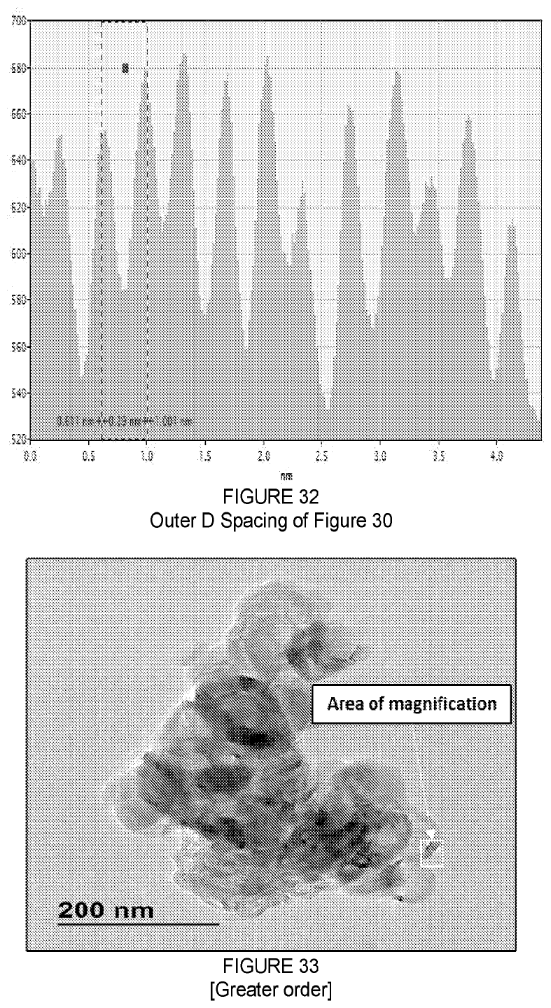

FIGS. 27-30 depict TEM images of the product from the 2000° C. thermal treatment. The TEM images were produced using a HRTEM (High Resolution Transmission Electron Microsocopy) device. FIG. 27 is at a magnification of 300 thousand, FIG. 28 is at a magnification of 600 thousand, FIG. 29 is at a magnification of one million, and FIG. 30 is at a magnification of ten million.

FIG. 30, which is taken from the identified portion of the product depicted in FIGS. 28 and 29, shows how ordered the carbon atoms have become. Note the lines of graphitic carbon, showing significantly more ordered atomic carbon than the original carbon feedstock had.

FIGS. 31 and 32 graph the inner and outer D spacing (respectively) of the 2000° C. treated feedstock depicted in FIG. 30. This tighter D spacing after treatment also shows increased order of C atoms. Further testing showed improved conductivity of the carbon product, which also indicates a greater ordering of the carbon atoms in the 2000° C. treated carbon product.

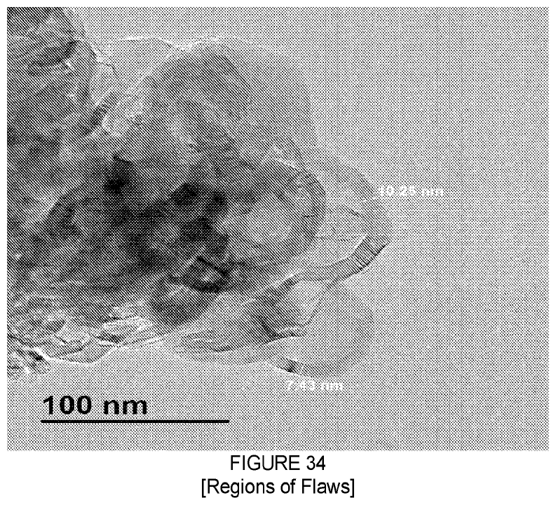

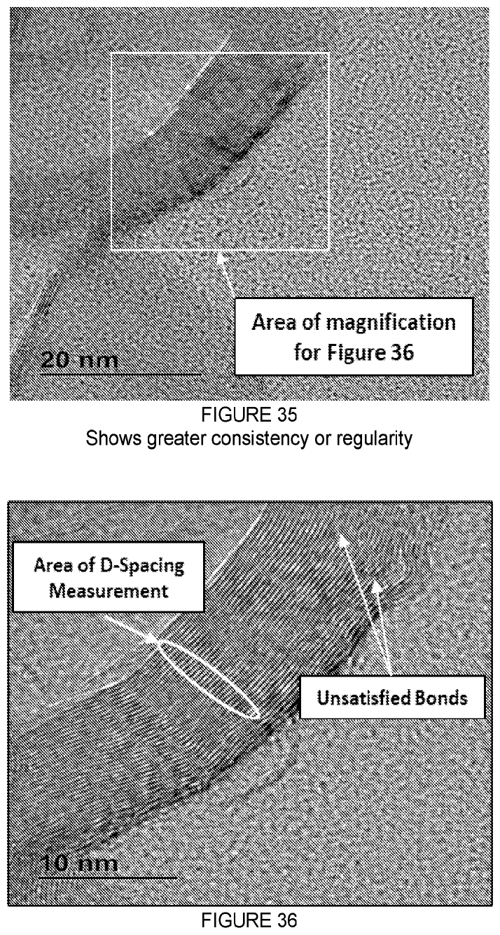

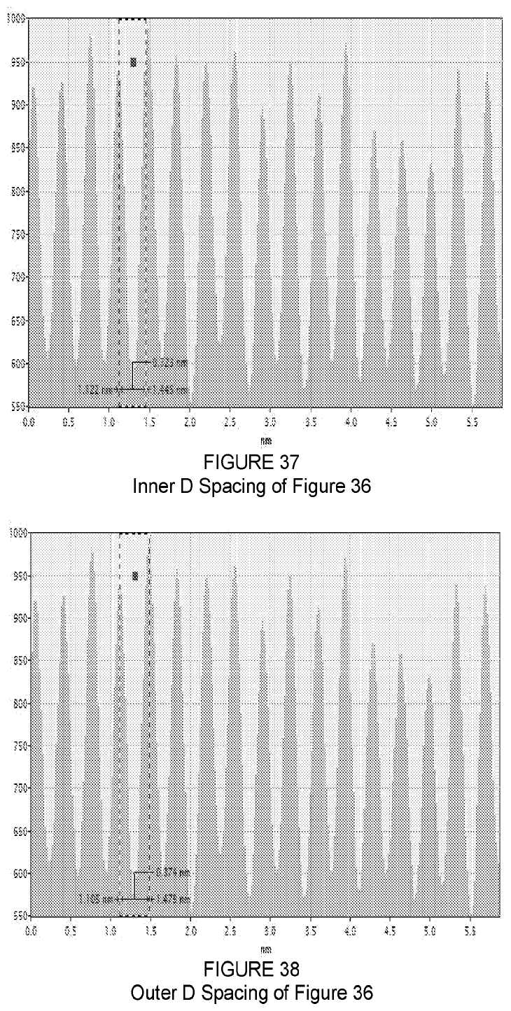

FIGS. 33-36 depict TEM images of the product from the 2400° C. thermal treatment. The TEM images were produced using a HRTEM (High Resolution Transmission Electron Microscopy) device. FIG. 33 is at a magnification of 600 thousand, FIG. 34 is at a magnification of one million, FIG. 35 is at a magnification of five million, and FIG. 36 is at a magnification of ten million.

FIG. 35, which is taken from the identified portion of the product depicted in FIG. 33, and FIG. 36, which is from the identified portion of FIG. 35, each show how ordered the carbon atoms have become. Note the relatively strong lines of graphitic carbon, showing significantly more ordered atomic carbon than the original carbon feedstock had.

FIGS. 37 and 38 graph the inner and outer D spacing (respectively) of the 2400° C. treated feedstock depicted in FIG. 36. This D spacing after treatment also shows increased order of carbon atoms; in particular, note the regularity of the D spacing, which is significantly improved even over the D spacing of the 2000° C. treated feedstock.

The Chart below shows the surface area, density, conductivity, resistivity, EDS (energy dispersive spectroscopy), and TGA (thermographic analysis) for each of the three experimental products.

| | Surface | | | | | | |
|---|---|---|---|---|---|---|---|
| | Area | Density | Density | | | | |
| Temperature | BET | Tap | TRUE | Conductivity | Resistivity | TGA | EDS |
| ° C. | m²/g | g/cc | g/cc | S/cm | Ω | % | % |
| 588 | 259.3371 | 0.199 | 1.697 | 11.77 | 0.1179 | 9.308 | 12.05 |
| 1600 | 125.3758 | 0.2582 | 1.426 | 43.81 | 0.02808 | 9.302 | 8.81 |
| 2000 | 86.7688 | 0.295 | 1.453 | 45.1 | 0.02217 | 4.475 | 8.15 |
| 2400 | 51.901 | 0.3069 | 1.586 | 56.1 | 0.01841 | 0.8892 | 0.14 |

Chart

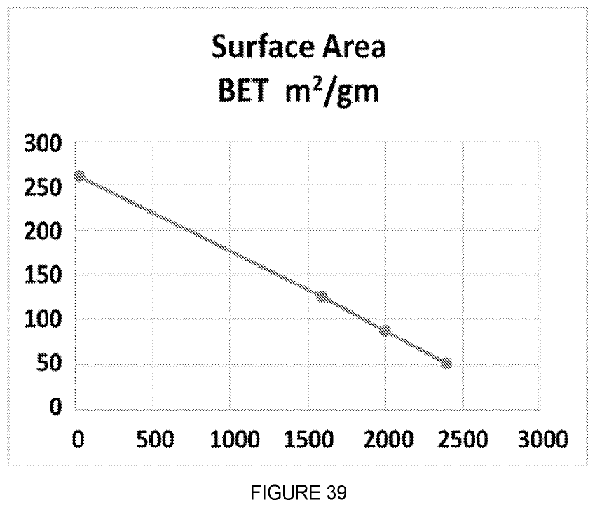

FIG. 39 graphs the surface area in square meters per gram of the carbon feedstock (left-most dot or circle) and the 1600° C., 2000° C., and 2400° C. carbon products from the experiments. In addition, the iron content of the 2400° C. treated feedstock product was significantly reduced. As the BET goes down (that is, for the higher temperature samples), this indicates a more graphitic morphology of the carbon. It appears that, were experiments conducted at even higher temperatures, the carbon product would likely become even more graphitic.

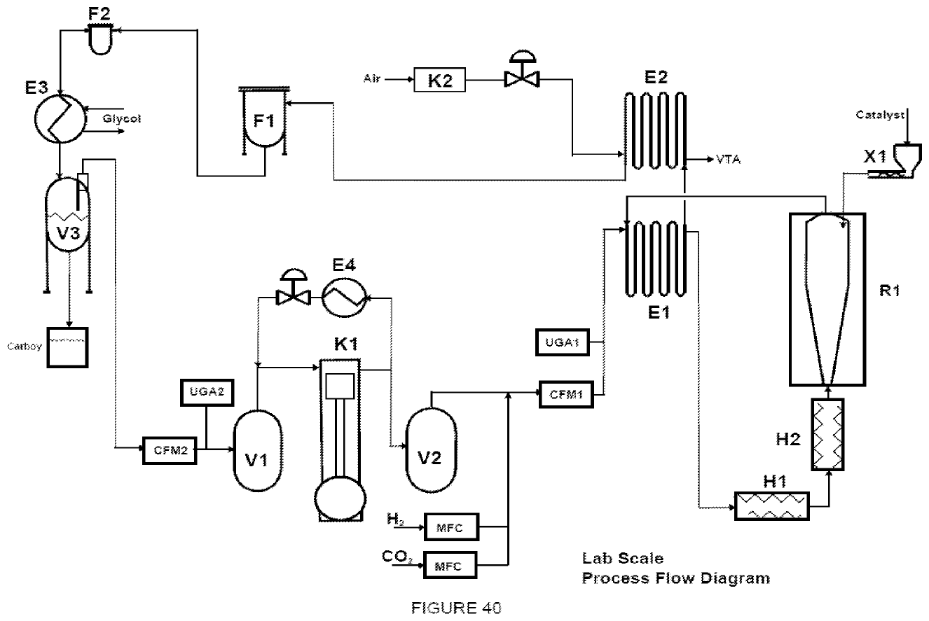

The carbon used for graphitization according to the present process may be made by different processes, but a preferred process is described herein. FIG. 40 shows a schematic representation of an exemplary process flow diagram. The various elements shown in FIG. 40 are labeled as:

| | |
|---|---|
| MFC | Mass Flow Controller |
| CFM | Coriolis Flow Meter |
| UGA | Universal Gas Analyzer |
| VTA | Vent to Atmosphere |
| X1 | Catalyst Feeder |
| E1 | Heat Exchanger |
| E2 | Heat Exchanger |
| H1 | Tube Furnace |
| H2 | Tube Furnace |
| R1 | Fluidized Bed Reactor |
| K2 | Air Compressor |
| F1 | Bag Filter |
| F2 | Fine Particulate Guard Filter |
| E3 | Glycol Heat Exchanger |
| V3 | Condensation Tank |
| V1 | Pressure Vessel Low Pressure |
| V2 | Pressure Vessel High Pressure |
| E4 | Heat Exchanger |
| K1 | Recirculation Compressor |

As depicted in FIG. 40, H₂ and CO₂ enter the process through designated Mass Flow Controllers (MFC). The amount of gases entering the system is controlled to maintain a gas composition within the reaction process and can be 0.1 standard liters per minute (“sl/m”) to 40 sl/m H2 and 2.0 slim to 38 sl/m CO₂. The CO₂ and H₂ enter the process before (upstream of) the Coriolis Flow Meter (CFM) where the mass balance is measured. The Universal Gas Analyzer (UGA1) measures the reaction gas composition prior as the gases pass by on the way to the reactor R1.

The gas composition entering the reaction process has a hydrogen content varying between 2% and 89.2%, a CO₂ content varying between 2% to 60%, a CH₄ content varying between. 05% to 65.7%, and a CO content varying between 5% to 60%. These reaction gases enter into the E1 tube-in-tube heat exchanger outside tube where the gases are preheated by the hot gases coming out of the reactor R1, which typically measure from 340° C. and 550° C. The heat exchangers E1 and E2 and the piping up to the fluidized bed reactor R1 are made from Inconel®—for example, product name HASTELLOY®.

The catalyst material typically comprises less than about 22 percent by weight (wt %) chromium, and less than about 14 wt % nickel (often less than about 8 wt % nickel). In some embodiments, the catalyst material comprises 316L stainless steel. 316L stainless steel comprises from about 16 wt % chromium to about 18.5 wt % chromium, and from about 10 wt % nickel to about 14 wt % nickel.

As the reaction gases pass through to the reactor R1, the Sabatier process starts, because the CO₂ and the H₂ react in the presence of the nickel in the metallurgy of the piping used in the construction of the heat exchanger. That is, the process is:

$${CO}_{2} + 4 {H}_{2} {\to }_{pressure}^{400 ° C .} {CH}_{4} + 2 {H}_{2} O$$

CO₂+4H₂→CH₄+2H₂O(under pressure and temperature of 400° C.).

The preheated gases leave the heat exchange tube E1 and then pass through H1 and H2 Tube Furnaces to heat the gas mixture up to 340° C. and 715° C., at which temperature when the carbon oxides and methane pass into the fluidized bed reactor vessel R1, they are converted to solid carbons and water in the presence of the iron catalyst of the reactor vessel R1. These carbons can be a blend of graphitic carbons and pyrolytic carbons. The ratio of these carbons can be varied by controlling the methane percentage within the reactor. Within the fluidized bed reactor R1, the Boudouard reaction—where the CO₂ is converted to CO—accounts for the CO presence in the reaction gas mixture. Pyrolytic carbons are formed by the conversion of methane to solid carbon and hydrogen in this portion of the reaction. Graphitic carbons are produced by occurrence of the Bosch reaction CO₂+2H₂↔C(s)+H₂O

A catalyst feeder X1 deposits the catalyst into the reactor R1. Various grades of the catalyst material may be used. For example, the catalyst material may be a grade of an iron-, chromium-, molybdenum-, cobalt-, tungsten-, or nickel-containing alloy or superalloy. Such materials are commercially available from numerous sources, such as from Special Metals Corp., of New Hartford, New York, under the trade name INCONEL®, or from Haynes, Int'l, Inc., of Kokomo, Indiana, under the trade name HASTELLOY® (e.g., HASTELLOY® B-2, HASTELLOY® B-3, HASTELLOY® C-4, HASTELLOY® C-2000, HASTELLOY® C-22, HASTELLOY® C-276, HASTELLOY® G-30, HASTELLOY® N, or HASTELLOY® W) or stainless steel.

In the present examples using the 0.3 ton per month (of carbon produced) reactor, the catalyst feeder X1, was loaded with iron catalyst FeC, Fe₂O₃ or Fe₃O₄ and the feed rate of the irons into the reactor vessel was from 5 grams per hour to 50 grams per hour. As the carbons were formed within the reactor vessel R1, various morphologies were produced by controlling the residence time in the reactor, for example, by converting carbon fiber to coke and blends thereof. Residence time was controlled by the flow rate of, typically, 40 sl/m to 215 sl/m of reaction gases through the reactor R1. The resulting carbon products were then carried out of the reactor, entrained in the gas stream. The reaction gases exited the reactor R1 and entered the inner tube of the heat exchanger E1 where the reaction gases (and carbon) exiting from the reactor were used to preheat the reaction gases going from Coriolis Flow Meter CFM1 to tube furnaces H1 and H2.

The catalyst material may comprise stainless steel, in which case the catalyst typically comprises less than about 22 percent by weight (wt %) chromium, and less than about 14 wt % nickel (often less than about 8 wt % nickel). In some embodiments, the catalyst material comprises 316L stainless steel. 316L stainless steel comprises from about 16 wt % chromium to about 18.5 wt % chromium, and from about 10 wt % nickel to about 14 wt % nickel.

Compressed air from air compressor K2 is used, at a controlled rate, to cool the reaction gases (and carbon) exiting the heat exchanger E1, to avoid heat damage to the bag filter media used in the bag filter housing F1. Reaction gases (and carbon) exiting from heat exchanger E1 enter heat exchanger E2, passing through the inner tube of heat exchanger E2 while the cooling air from compressor K2 passes through the outer tube of heat exchanger E2. The compressed air, used to cool the reaction gases, is vented to the atmosphere (VTA) to disperse its heat. The reaction gases passing through the inner tube of heat exchanger E2 must be kept hot enough to prevent water from prematurely condensing out of the reaction gases, that is, before the reaction gases pass into heat exchanger E3. A fine particulate guard filter F2 captures any carbon that was not captured upstream by the bag filter F1. The water of reaction is as a result of the reverse water gas shift reaction.

The gas stream then flows from bag filter F1 downstream to glycol heat exchanger E3. From the heat exchanger E3, the reaction gases pass into condensation tank V3, where a reverse water gas shift is allowed to happen. Water thus collected in condensation tank V3 is pumped off to a carboy for disposal or use. The gases leaving condensation tank V3 then pass through a second Coriolis Flow Meter CFM2 where the mass balance is measured and past the universal gas analyzer UGA2 that is used for determining the gas composition. The measurements from the first set of Coriolis flow meter CFM1 and universal gas analyzer UGA1 and the second set of Coriolis flow meter CFM2 and universal gas analyzer UGA2 allow the determination of the gas conversion through the process. The measurements during the above-described experiments showed that the carbon conversion rate of the process ranged from 6.3 grams per hour to 1480.2 grams per hour.

The gases then flow to pressure vessel low pressure V1, the low-pressure side of the recirculation compressor K1 that is used to circulate the gases through the closed loop process. The gases are then pressurized by the compressor K1 to the required process pressure. The pressure vessel high pressure V2 serves as a stabilizer, removing the gas pulses coming from the compressor K1. A heat exchanger E4 is used to cool the gases from the compressor high-pressure side to protect the flow valve used for system pressure control.

### Example

Carbon dioxide and hydrogen were fed into a continuous flow reactor with the temperature set at 590° C. and with the reactor set to maintain a pressure of 50 psi. The CO₂ feed rate was set at 2.2 sl/m and H₂ set at 8.7 sl/m. The iron catalyst feed rate was set at 5 grams per hour. These conditions were allowed to run for 112 hours. During this time the reactor average gas composition was 9.2% H₂, 6.1% CH₄, 38.75% CO and 47.2% CO₂ with the gas composition set to flow through the reactor at 128 sl/m. The reaction produced 7.74 kg of carbon pitch (or coke) over the 112 hours of run time.

Thus, according to the present disclosure, treatment of carbon feedstock can be customized to produce a carbon product with desired characteristics. That is, carbon produced as described herein can be thermally treated to take out impurities as well as increase the graphitization of the carbon product. This results in increased conductivity of the carbon product.

Furthermore, these processes use carbon dioxide (such as that scrubbed from refinery flue gases) to make a carbon pitch for production of synthetic graphite, or a “dry” coke from those very refinery reactor flue gases. This “dry” carbon pitch or coke is produced not from petroleum tar or other such “wet” feedstock as was previously known. Furthermore, that “dry” coke could then be used to make synthetic graphite and typically has a significant carbon fiber content, which fiber content may be increased or reduced based on the operating parameters of the production.

Although particular embodiments of the present invention have been described, those of skill in the art will appreciate that various modifications and changes may be made by those skilled in the art without departing from the spirit and scope of the invention. The present invention may be embodied in other specific forms without departing from its structures, methods, or other essential characteristics as broadly described herein and claimed hereinafter. The described embodiments are to be considered in all respects only as illustrative, and not restrictive.

1. A method for producing synthetic graphite comprising the steps of:
feeding a reaction mixture comprising carbon dioxide and hydrogen at a first predetermined feed rate into a reactor at a predetermined temperature and a predetermined pressure, a portion of the reaction mixture being converted to methane as the reaction mixture is being fed into the reactor;
feeding a catalyst into the reactor at second predetermined feed rate;
maintaining the reaction process for a predetermined time to produce a solid carbon reaction product;
removing the solid carbon reaction product from the reactor and cooling the solid carbon reaction product;
condensing out any water and other gaseous impurities from the solid carbon reaction product;
placing a quantity of the solid carbon reaction product into a reaction vessel;
loading the reaction vessel into a vacuum furnace equipped with a vacuum pump;
closing the furnace and beginning to heat the furnace to increase the temperature of the reaction vessel;
starting the vacuum pump;
increasing the furnace temperature at a first predetermined rate;
maintaining the temperature of the reaction vessel at a first predetermined temperature for a first predetermined time;
increasing the furnace temperature at a second predetermined rate until the reaction vessel temperature reaches a second predetermined temperature;
maintaining the reaction vessel temperature at the second predetermined temperature for a second predetermined time;
cooling the reaction vessel at a rate sufficient to ensure that the processed solid carbon reaction product will not oxidize upon opening of the furnace;
opening the furnace and allowing the reaction vessel to cool to a handling temperature; and
removing the processed solid carbon reaction product from the reaction vessel.

2. The method of claim 1 further comprising the step of introducing a flow of a relatively inert gas into the furnace at a predetermined rate of flow.

3. A method for treating a mixture of amorphous and fibrous carbon to increase order and conductivity comprising the steps of:
placing a quantity of carbonaceous material comprising amorphous or fibrous forms of carbon or a combination thereof into a reaction vessel;
loading the reaction vessel into a vacuum furnace equipped with a vacuum pump; closing the furnace and beginning to heat the furnace to increase the temperature of the reaction vessel;
starting the vacuum pump;
increasing the furnace temperature at a first predetermined rate;
maintaining the temperature of the reaction vessel at a first predetermined temperature for a first predetermined time;
increasing the furnace temperature at a second predetermined rate until the reaction vessel temperature reaches a second predetermined temperature;
maintaining the reaction vessel temperature at the second predetermined temperature for a second predetermined time;
cooling the reaction vessel at a rate sufficient to ensure that processed carbonaceous material will not oxidize upon opening of the furnace;
opening the furnace and allowing the reaction vessel to cool to a handling temperature; and
removing the carbonaceous material from the reaction vessel.

4. The method of claim 3 wherein the first predetermined rate is approximately 20° C. per minute.

5. The method of claim 3 wherein the first predetermined temperature is approximately 2000° C. and the first predetermined time is approximately 30 minutes.

6. The method of claim 3 wherein the second predetermined temperature is approximately 2400° C. and the second predetermined rate is approximately 20° C. a minute.

7. The method of claim 3 wherein the second predetermined time is approximately 4 hours.

8. The method of claim 3 further comprising the step of introducing a flow of a relatively inert gas into the furnace at a predetermined rate of flow.

9. A method for producing a carbon pitch comprising the steps of:
feeding a reaction mixture comprising carbon dioxide and hydrogen at a first predetermined feed rate into a reactor at a predetermined temperature and a predetermined pressure, a portion of the reaction mixture being converted to methane as the reaction mixture is being fed into the reactor;
feeding a catalyst into the reactor at second predetermined feed rate;
maintaining the reaction process for a predetermined time to produce a predetermined amount of carbon pitch reaction product;
removing the reaction product from the reactor and cooling the reaction product;
condensing out any water and other gaseous impurities from the reaction product; and
collecting the reaction product.

10. The method of claim 9 wherein the reaction mixture has a hydrogen content between 2% and 89.2% and a carbon dioxide content between 2% to 60%.

11. The method of claim 9 wherein the methane converted comprises between 0.5% and 65.7% of the reaction mixture after the reaction mixture has been fed into the reactor.

12. The method of claim 9 wherein the predetermined temperature is between 340° C. and 540° C.

13. The method of claim 9 wherein the predetermined pressure is between 14 and 610 pounds per square inch.

14. The method of claim 9 wherein the predetermined time is between 1 and 1250 hours.
